(19) United States
(12) Patent Application Publication
(10) Pub. No.: US 2009/0270956 A1
(43) Pub. Date: Oct. 29, 2009

(54) IMPLANTABLE MEDICAL LEAD CONFIGURED FOR IMPROVED MRI SAFETY

(75) Inventors: Abhi Vase, San Jose, CA (US); Dorab N. Sethna, Valencia, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/110,150

(22) Filed: Apr. 25, 2008

(51) Int. Cl. A61N 1/00

(52) U.S. Cl. 607/116

(57) ABSTRACT

Disclosed herein is an implantable medical lead for coupling to an implantable pulse generator and configured for improved MRI safety. In one embodiment, the lead includes a tubular body, an electrode, an electrical conductor, and a shield layer. The tubular body includes a proximal end and a distal end. The electrode is operably coupled to the tubular body near the distal end. The electrical conductor extends distally through the body from the proximal end and electrically connects to the electrode. The shield layer extends through the tubular body between the proximal and distal ends. The shield layer is configured to reduce an amount of current induced in the electrical conductor when present in an electromagnetic field as compared to the current that would be induced in the electrical conductor absent the shield layer.

### CROSS-REFERENCE TO RELATED APPLICATION

The present application contains subject matter that is related to U.S. patent application Ser. No. 11/932,030, filed Oct. 31, 2007, entitled “Implantable Medical Lead Configured for Improved MRI Safety” (Attorney Docket A07P1164).

### FIELD OF THE INVENTION

The present invention relates to medical methods and apparatus. More specifically, the present invention relates to implantable medical leads and methods of manufacturing and utilizing such leads.

### BACKGROUND OF THE INVENTION

Existing implantable medical leads for use with implantable pulse generators, such as neurrostimulators, pacemakers, defibrillators or implantable cardioverter defibrillators (“ICD”), are prone to heating due to induced current when placed in the strong magnetic (static, gradient and RF) fields of a magnetic resonance imaging (“MRI”) machine. The heating and induced current are the result of the lead acting like an antenna in the magnetic fields generated during a MRI. Heating and induced current in the lead may result in incorrect stimulation or deterioration of stimulation thresholds or, in the context of a cardiac lead, even increase the risk of cardiac tissue perforation.

Over fifty percent of patients with an implantable pulse generator and implanted lead require, or can benefit from, a MRI in the diagnosis or treatment of a medical condition. MRI modality allows for flow visualization, characterization of vulnerable plaque, non-invasive angiography, assessment of ischemia and tissue perfusion, and a host of other applications. The diagnosis and treatment options enhanced by MRI is only going to grow over time. For example, MRI has been proposed as a visualization mechanism for lead implantation procedures.

There is a need in the art for an implantable medical lead configured for improved MRI safety. There is also a need in the art for methods of manufacturing and using such a lead.

### SUMMARY

Disclosed herein is an implantable medical lead for coupling to an implantable pulse generator and configured for improved MRI safety. In one embodiment, the lead includes a tubular body, an electrode, an electrical conductor, and a shield layer. The tubular body includes a proximal end and a distal end. The electrode is operably coupled to the tubular body near the distal end. The electrical conductor extends distally through the body from the proximal end and electrically connects to the electrode. The shield layer extends through the tubular body between the proximal and distal ends. The shield layer is configured to reduce an amount of current induced in the electrical conductor when present in an electromagnetic field as compared to the current that would be induced in the electrical conductor absent the shield layer.

Disclosed herein is an implantable medical lead for coupling to an implantable pulse generator and configured for improved MRI safety. In one embodiment, the lead includes a tubular body, an electrode, and an electrical conductor, and a tubular shield layer. The tubular body includes a proximal end and a distal end. The electrode is operably coupled to the tubular body near the distal end. The electrical conductor extends distally through the body from the proximal end and electrically connects to the electrode. The tubular shield layer longitudinally extends through the tubular body between the proximal and distal ends and may be electrically grounded.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of a lead and a pulse generator for connection thereto.

FIG. 2 is an isometric view of a longitudinal segment of the lead tubular body in the vicinity of arrow A in FIG. 1, wherein layers of the body have been removed in a stepped fashion to depict the various layers.

FIGS. 3 and 4 are diagrams illustrating the electrical connections between the various components of the lead and the pulse generator.

FIG. 5 is the same view depicted in FIG. 2, except of another embodiment of the lead body.

FIG. 6 is the same view depicted in FIG. 2, except of another embodiment of the lead body.

FIGS. 7 and 8 are generally the same view as depicted in FIGS. 3 and 4, respectively, except having a different shield configuration.

FIG. 9 is the same view depicted in FIG. 6, except of another embodiment of the lead body.

FIGS. 10 and 11 are both the same view depicted in FIG. 6, except of other embodiments of the lead body.

FIGS. 12 is the same view depicted in FIG. 6, except of another embodiment of the lead body.

### DETAILED DESCRIPTION

Disclosed herein is an implantable medical lead 10 configured for improved MRI safety. In various embodiments, the lead 10 includes coils and/or shields configured to reduce, if not totally eliminate, the potential for MRI induced currents and heating in conductors extending through the lead body to electrodes, such as those used for pacing, sensing and/or defibrillation.

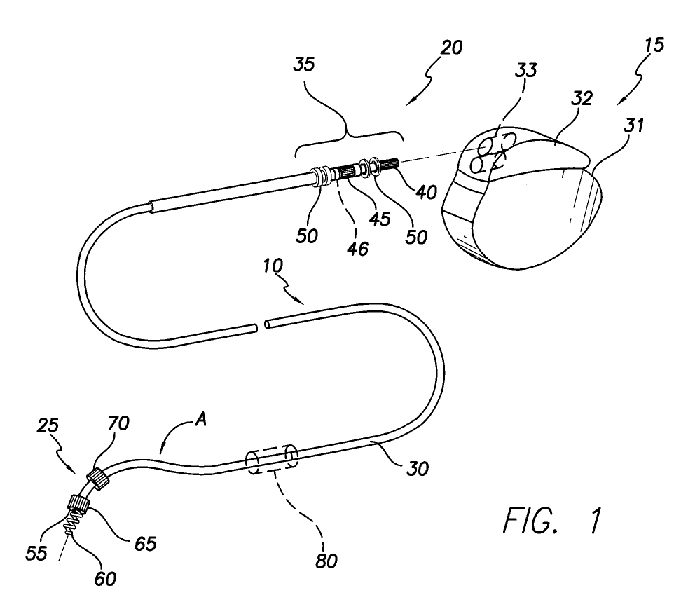

For an overview discussion regarding an embodiment of a lead 10 configured for improved MRI safety, reference is made to FIG. 1, which is an isometric view of such a lead 10 and a pulse generator 15 for connection thereto. As shown in FIG. 1, the pulse generator 15, which may be a neurrostimulator, pacemaker, defibrillator or ICD, includes a housing 31 and a header 32. The housing encloses the electrical components of the pulse generator. The header is mounted on the housing and includes lead receiving receptacles 33 for connecting one or more leads to the pulse generator.

As illustrated in FIG. 1, in one embodiment, the lead 10 includes a proximal end 20, a distal end 25 and a tubular body 30 extending between the proximal and distal ends. The proximal end 20 includes a lead connective end 35 having a pin contact 40, a first ring contact 45, a second ring contact 46, which is optional, and sets of spaced-apart radially projecting seals 50. In other embodiments, the lead connective end will include a greater or lesser number of contacts and will include the same or different types of seals. The lead connective end 35 is received in a lead receiving receptacle 33 of the pulse generator 15 such that the contacts 40, 45, 46 electrically contact corresponding electrical terminals within the receptacle, and the seals 50 seals prevent the ingress of body fluids into the receptacle.

As depicted in FIG. 1, in one embodiment, the lead distal end 25 includes a distal tip 55, an anchor 60, a tip electrode 65, and a ring electrode 70. The anchor 60 is extendable from an orifice in the distal tip 55. The tip electrode 65 forms the distal tip 55 of the lead body 30, and the ring electrode 70 extends about the circumference of the lead body 30 proximal of the tip electrode 65. In other embodiments, there will be a greater or lesser number of electrodes 65, 70 in similar or different configurations. Also, the anchor 60 may or may not have other configurations and may or may not also serve as an electrode.

As indicated in FIG. 1, the lead 10 includes an optional defibrillation coil 80, which extends about the circumference of the lead body 30. The defibrillation coil 80 is located proximal of the ring electrode 70.

In one embodiment, the tip electrode 65 is in electrical communication with the pin contact 40 via electrical conductors, the ring electrode 70 is in electrical communication with the ring contact 45 via other electrical conductors, and the defibrillation coil 80 is in electrical communication with the second ring contact 46 via yet other conductors. The various conductors extend through the lead body 30 and are described later in this Detailed Description.

But for the novel lead body and conductor configurations discussed below, the conductors could act as an antenna in the magnetic field of an MRI. As a result, current could be induced in the conductors, causing the conductors and the electrodes connected thereto to heat and potentially damage the lead and/or tissue contacting the electrodes.

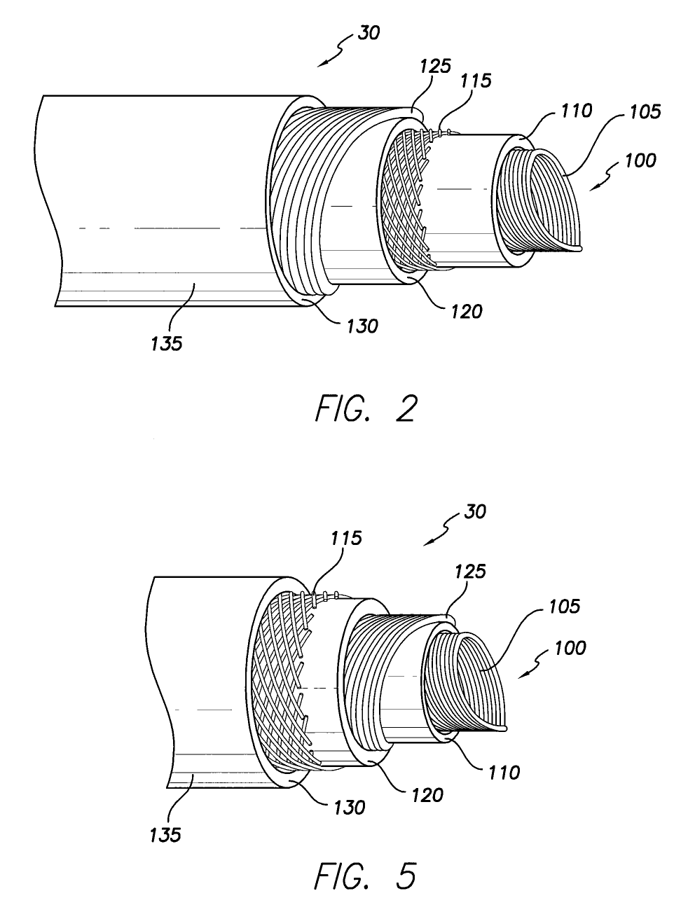

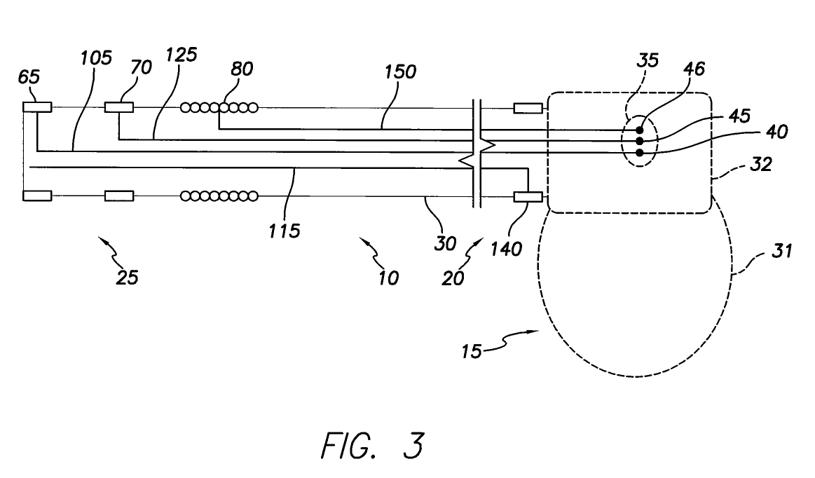

For a discussion of a first embodiment of the lead 10 configured to reduce, if not totally eliminate, the current induction and heating caused in lead conductors subjected to MRI, reference is made to FIGS. 2-4. FIG. 2 is an isometric view of a longitudinal segment of the lead tubular body 30 in the vicinity of arrow A in FIG. 1, wherein layers of the body 30 have been removed in a stepped fashion to depict the various layers. FIGS. 3 and 4 are diagrams illustrating the electrical connections between the various components of the lead 10 and the pulse generator 15.

As shown in FIG. 2, in one embodiment, the lead body 30 includes a central lumen 100, an inner or lumen conductor coil 105, an inner insulation layer 110, a mesh or shield layer 115, an intermediate insulation layer 120, an outer conductor coil 125, and an outer insulation layer 130.

As illustrated in FIG. 2, in one embodiment, the inner conductor coil 105 extends helically through the lead body 30 such that the inner conductor coil 105 is generally tubular or cylindrical in shape and defines the lumen 100. The lumen 100 is configured such that a guidewire or stylet can be extended through the lumen. As can be understood from FIGS. 1, 3 and 4, the proximal end of the inner conductor coil 105 is electrically coupled to the pin contact 40, and the distal end of the inner conductor coil 105 is electrically coupled to the tip electrode 65. The inner conductor coil 105 is formed of an electrically conductive material such as MP35N, 35NLT, DFT, etc. The inner conductor coil 105 is formed of a single filar or multiple filars. The filar or fillars forming the inner conductor coil 105 may have a round cross-section with an OD ranging from approximately 0.001 inches to 0.006 inches, or may have a rectangular cross-section of roughly (0.00075 inches×0.005 inches) to (0.0025 inches×0.005 inches).

As depicted in FIG. 2, in one embodiment, the inner insulation layer 110 extends about the outer circumferential surface of the inner conductor coil 105. The inner insulation layer 110 extends generally the full length of the tubular body 30 between the proximal and distal ends 20, 25 of the body 30. The inner insulation layer 110 is generally tubular or cylindrical in shape. The inner insulation layer 110 is formed from an electrically insulative material such as silicone, polyurethane, PTFE, silicone rubber-polyurethane-copolymer ((“SPC”), also known as Optim, Hemoflex, Elasteon), etc. The inner insulation layer 110 has a wall thickness of between approximately 0.0005 inches and approximately 0.01 inches.

As shown in FIG. 2, in one embodiment, the shield layer 115 extends about the outer circumferential surface of the inner insulation layer 110. The shield layer 115 extends generally the full length of the tubular body 30 between the proximal and distal ends 20, 25 of the body 30. The shield layer 115 is generally tubular or cylindrical in shape. The shield layer 115 has a mesh, weave or-helically coiled conductor configuration and is formed from an electrically conductive material such as stainless steel 316L, MP35N, 35NLT, DFT, etc. In one embodiment, the wires or conductors forming the shield layer 115 may have a round cross-section of OD ranging from approximately 0.001 inches to 0.006 inches, or may have a rectangular cross-section of roughly (0.00075 inches×0.005 inches) to (0.0025 inches×0.005 inches). The braid mesh may have a pick count of 20 picks per inch (ppi) to 80 picks per inch, and may be made with between 8 and 32 carrier-wires.

In one embodiment, the shield layer 115 is entirely encapsulated via electrical insulation such that the shield layer 115 is entirely electrically isolated from any conductive features of the lead 10, pulse generator 15 or patient. In other words, the shield layer 115 is not electrically grounded.

In other embodiments, the shield layer 115 is electrically coupled to a ground such as the pulse generator 15 or the patient. For example, as depicted in FIG. 3, in one embodiment, the proximal end 20 of the lead body 30 includes a ground electrode 140. In one embodiment, the ground electrode 140 is in the form of a ring extending about the outer circumferential surface of the lead body 30. The proximal end of the shield layer 115 is electrically coupled to the ground electrode 140, and the rest of the shield layer 115 is electrically insolated from the conductive features of the lead 10, pulse generator 15 and patient via electrical insulation. In one embodiment, the ground electrode 140 is located on the proximal end 20 of the lead body 30 just distal of the lead connective end 35 such that the ground electrode 140 is located just outside of the header 32 of the pulse generator 15 when the lead connective end 35 is received in the receptacle 33 of the header 32. When the lead 10 and pulse generator 15 are implanted in the patient, the ground electrode 140 is placed in electrical contact with the patient tissue surrounding the implanted pulse generator 15.

As depicted in FIG. 4, in one embodiment, the proximal end of the shield layer 115 is electrically coupled to a contact ring 142 on the lead connective end 35, and the rest of the shield layer 115 is electrically insolated from the conductive features of the lead 10, pulse generator 15 and patient via electrical insulation. When the lead connective end 35 is received in the receptacle 33 of the header 32 to electrically couple the lead 10 to the pulse generator 15, the contact ring 142 is electrically coupled to a ground 145 in the pulse generator 15.

As indicated in FIG. 2, in one embodiment, the intermediate insulation layer 120 extends about the outer circumferential surface of the shield layer 115. The intermediate insulation layer 120 extends generally the full length of the tubular body 30 between the proximal and distal ends 20, 25 of the body 30. The intermediate insulation layer 120 is generally tubular or cylindrical in shape. The intermediate insulation layer 120 is formed from an electrically insulative material such as silicone rubber, SPC, PTFE, polyurethane, etc. The intermediate insulation layer 120 has a wall thickness of between approximately 0.0005 inches and approximately 0.01 inches.

As shown in FIG. 2, in one embodiment, the outer conductor coil 125 extends helically about the outer circumferential surface of the intermediate insulation layer 120. The outer conductor coil 125 is generally tubular or cylindrical in shape. As can be understood from FIGS. 1, 3 and 4, the proximal end of the outer conductor coil 125 is electrically coupled to the ring contact 45, and the distal end of the outer conductor coil 125 is electrically coupled to the ring electrode 70. The outer conductor coil 125 is formed of an electrically conductive material such as MP35N, 35NLT, DFT, etc. The outer conductor coil 125 is formed of a single filar or multiple filars. The filar or fillars forming the outer conductor coil 125 may have a round cross-section with an OD ranging from approximately 0.001 inches to 0.006 inches, or may have a rectangular cross-section of roughly (0.00075 inches×0.005 inches) to (0.0025 inches×0.005 inches).

As illustrated in FIG. 2, in one embodiment, the outer insulation layer 130 extends about the outer circumferential surface of the outer conductor coil 125. The outer insulation layer 130 extends generally the full length of the tubular body 30 between the proximal and distal ends 20, 25 of the body 30. The outer insulation layer 130 is generally tubular or cylindrical in shape. The outer insulation layer 130 is formed from an electrically insulative material such as silicone rubber, SPC, PTFE, polyurethane, etc. The outer insulation layer 130 has a wall thickness of between approximately 0.004 inches and approximately 0.01 inches.

In one embodiment, the inner and outer conductor coils 105,125 are helically wound in the same direction. However, in other embodiments, as can be understood from FIG. 2, the inner and outer conductor coils 105, 125 are helically wound in opposite directions. In either case, the shield layer 115 is located between the layers of the inner and outer conductor coils 105, 125. The shield layer 115 acts as a shield to the prevent the RF energy from a MRI from inducing current in the inner conductor coil 105. For example, any electric fields induced within the shield layer will oppose each other and cancel out by virtue of the directionality of the currents induced in the shield layer. As a result, the MRI field and any fields induced by the MRI field will be invisible to the conductor coil 105 that lays within the shield layer.

As can be understood from FIGS. 3 and 4, a conductor 150 extends through the tubular body 30 to electrically couple the defibrillation coil 80 to the contact ring 46. In one embodiment, the conductor 150 is a helical conductor coil similar to those described with respect to the inner and outer conductor coils 105, 125. In other embodiments, the conductor 150 is a wire or cable formed of electrically conductive materials similar to those employed for the inner and outer conductor coils 105, 125.

FIG. 2 depicts inner and intermediate insulation layers 110, 120 electrically isolating the inner conductor coil 105 from the shield layer 115 and the outer conductor coil 125 from the shield layer 115. However, in other embodiments, one or more of the insulation layers 110, 120 can be eliminated where: one or more of the conductor coils 105, 125 and/or the shield layer 115 is coated or jacketed with an electrical insulation; or the wires or filars forming the conductor coils 105, 125 and/or the shield layer 115 are individually coated or jacketed with an electrical insulation. In other words, in one embodiment, one or more of the insulation layers 110, 120 can be eliminated where other steps are taken to electrically insulate the conductor coils 105, 125 from each other and/or from the shield layer 115.

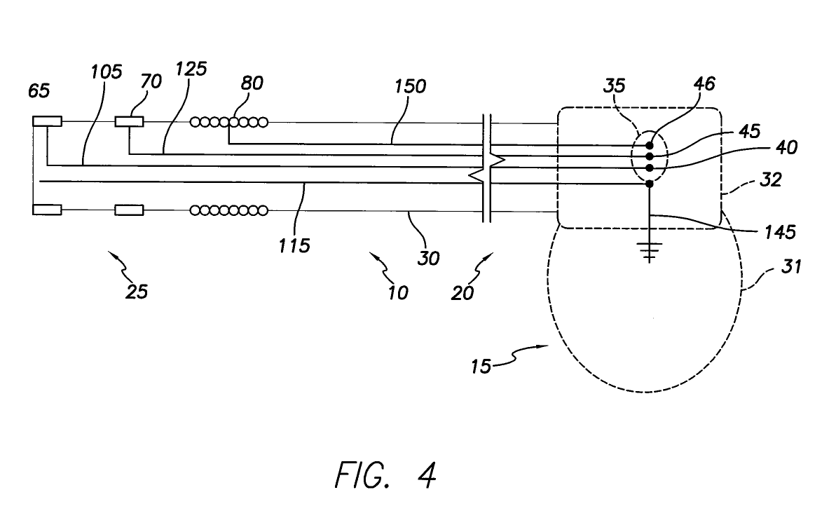

For a discussion of a second embodiment of the lead 10 configured to reduce, if not totally eliminate, the current induction and heating caused in lead conductors subjected to MRI, reference is made to FIG. 5, which is the same view depicted in FIG. 2, except of another embodiment of the lead body 30. As can be understood from a comparison of FIGS. 2 and 5, the lead body 30 depicted in FIG. 5 is generally the same as the lead body 30 depicted in FIG. 2, except the various layers of the lead body 30 are arranged in a different order. Specifically, as indicated in FIG. 5, the inner conductor coil 105 defines the lumen 100, the inner insulation layer 110 extends about the inner conductor coil 105, the outer conductor coil 125 extends about the inner insulation layer 110, the intermediate insulation layer 120 extends about the outer conductor coil 125, the shield layer 115 extends about the intermediate insulation layer 120, and the outer insulation layer 130 extends about the shield layer 115. Generally speaking, all other aspects of the embodiment depicted in, and discussed with respect to, FIGS. 1-4 are equally applicable to the embodiment depicted in FIG. 5.

In one embodiment, the inner and outer conductor coils 105, 125 are helically wound in the same direction. However, in other embodiments, as can be understood from FIG. 5, the inner and outer conductor coils 105, 125 are helically wound in opposite directions. In either case, the shield layer 115 is located outside the layers of the inner and outer conductor coils 105, 125. The shield layer 115 acts as a shield to prevent the RF energy from a MRI from inducing current in the inner and outer conductor coils 105, 125. For example, in one embodiment, the shield layer 115 can act as an antenna for the field energy that would otherwise induce current in the conductor coils 105, 125. For example, any electric fields induced within the shield layer will oppose each other and cancel out by virtue of the directionality of the currents induced in the shield layer. As a result, the MRI field and any fields induced by the MRI field will be invisible to the conductor coils 105, 125 that lay within the shield layer.

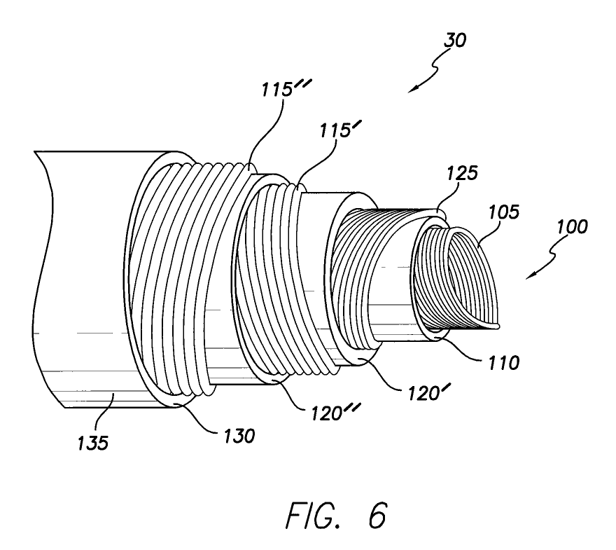

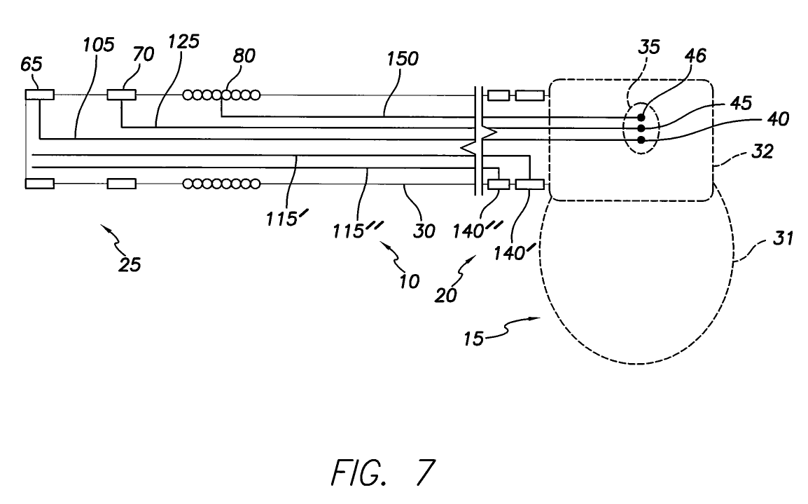

For a discussion of a third embodiment of the lead 10 configured to reduce, if not totally eliminate, the current induction and heating caused in lead conductors subjected to MRI, reference is made to FIGS. 6-8. FIG. 6 is the same view depicted in FIG. 2, except of another embodiment of the lead body 30. FIGS. 7 and 8 are generally the same view as depicted in FIGS. 3 and 4, respectively, except having a different shield configuration. As shown in FIG. 6, in one embodiment, the lead body 30 includes a central lumen 100, an inner or lumen conductor coil 105, an inner insulation layer 110, a first coil or shield layer 115′, a second coil or shield layer 115″, a first intermediate insulation layer 120′, a second intermediate insulation layer 120″, an outer conductor coil 125, and an outer insulation layer 130.

As illustrated in FIG. 6, in one embodiment, the inner conductor coil 105 extends helically through the lead body 30 such that the inner conductor coil 105 is generally tubular or cylindrical in shape and defines the lumen 100. The lumen 100 is configured such that a guidewire or stylet can be extended through the lumen. As can be understood from FIGS. 1, 7 and 8, the proximal end of the inner conductor coil 105 is electrically coupled to the pin contact 40, and the distal end of the inner conductor coil 105 is electrically coupled to the tip electrode 65. The inner conductor coil 105 is formed of an electrically conductive material such as MP35N, 35NLT, DFT etc. The inner conductor coil 105 is formed of a single filar or multiple filars. The filar or fillars forming the inner conductor coil 105 may have a round cross-section of OD ranging from approximately 0.001 inches to 0.006 inches, or may have a rectangular cross-section of roughly (0.00075 inches×0.005 inches) to (0.0025 inches×0.005 inches).

As depicted in FIG. 6, in one embodiment, the inner insulation layer 110 extends about the outer circumferential surface of the inner conductor coil 105. The inner insulation layer 110 extends generally the full length of the tubular body 30 between the proximal and distal ends 20, 25 of the body 30. The inner insulation layer 110 is generally tubular or cylindrical in shape. The inner insulation layer 110 is formed from an electrically insulative material such as Silicone, Optim (also known as SPC/Hemoflex/Elasteon etc.), PTFE, Polyurethane, etc. The inner insulation layer 110 has a wall thickness of between approximately 0.0005 inches and approximately 0.005 inches.

As shown in FIG. 6, in one embodiment, the outer conductor coil 125 extends helically about the outer circumferential surface of the inner insulation layer 110. The outer conductor coil 125 is generally tubular or cylindrical in shape. As can be understood from FIGS. 1, 7 and 8, the proximal end of the outer conductor coil 125 is electrically coupled to the ring contact 45, and the distal end of the outer conductor coil 125 is electrically coupled to the ring electrode 70. The outer conductor coil 125 is formed of an electrically conductive material such as MP35N. 35NLT, DFT, etc. The outer conductor coil 125 is formed of a single filar or multiple filars. The filar or filars forming the outer conductor coil 125 may have a round cross-section of OD ranging from approximately 0.001 inches to 0.006 inches, or may have a rectangular cross-section of roughly (0.00075 inches×0.005 inches) to (0.0025 inches×0.005 inches).

As indicated in FIG. 6, in one embodiment, the inner or first intermediate insulation layer 120′ extends about the outer circumferential surface of the outer conductor coil 125. The first intermediate insulation layer 120′ extends generally the full length of the tubular body 30 between the proximal and distal ends 20, 25 of the body 30. The first intermediate insulation layer 120′ is generally tubular or cylindrical in shape. The first intermediate insulation layer 120′ is formed from an electrically insulative material such as Silicone, Optim (also known as SPC/Hemoflex/Elasteon etc.), PTFE, Polyurethane, etc. The first intermediate insulation layer 120′ has a wall thickness of between approximately 0.0005 inches and approximately 0.010 inches.

As shown in FIG. 6, in one embodiment, the first or inner shield layer 115′ is a helically wound coil extending about the outer circumferential surface of the first intermediate insulation layer 120′. The first shield layer 115′ extends generally the full length of the tubular body 30 between the proximal and distal ends 20, 25 of the body 30. The first shield layer 115′ is generally tubular or cylindrical in shape. In one embodiment, the helically wound coil forming the first shield layer 115′ is formed of a single helically wound filar or wire or multiple helically wound filars or wires. In one embodiment, the filars or wires forming the first shield layer 115′ may have a round cross-section of OD ranging from approximately 0.001 inches to 0.006 inches, or may have a rectangular cross-section of roughly (0.00075 inches×0.005 inches) to (0.0025 inches×0.005 inches), and the wires or filars are formed from an electrically conductive material such as MP35N, 35NLT, Stainless Steel 316L, DFT, etc.

As indicated in FIG. 6, in one embodiment, the outer or second intermediate insulation layer 120″ extends about the outer circumferential surface of the outer first shield layer 115′. The second intermediate insulation layer 120″ extends generally the full length of the tubular body 30 between the proximal and distal ends 20, 25 of the body 30. The second intermediate insulation layer 120″ is generally tubular or cylindrical in shape. The second intermediate insulation layer 120″ is formed from an electrically insulative material such as Silicone, PTFE, Optim, Polyurethane, etc. The second intermediate insulation layer 120″ has a wall thickness of between approximately 0.0005 inches and approximately 0.010 inches.

As shown in FIG. 6, in one embodiment, the second or outer shield layer 115″ is a helically wound coil extending about the outer circumferential surface of the second intermediate insulation layer 120″. The second shield layer 115″ extends generally the full length of the tubular body 30 between the proximal and distal ends 20, 25 of the body 30. The second shield layer 115″ is generally tubular or cylindrical in shape. In one embodiment, the helically wound coil forming the second shield layer 115″ is formed of a single filar or wire or multiple filars or wires, which are helically wound in an opposite direction from the helically wound filars or wires of the first shield layer 115′. In one embodiment, the filars or wires forming the second shield layer 115″ may have a round cross-section of OD ranging from approximately 0.001 inches to 0.006 inches, or may have a rectangular cross-section of roughly (0.00075 inches×0.005 inches) to (0.0025 inches×0.005 inches), and the wires or filars are formed from an electrically conductive material such as MP35N, 35NLT, Stainless Steel 316L, DFT, etc.

In one embodiment, the shield layers 115′, 115″ are entirely encapsulated via electrical insulation such that the shield layers 115′, 115″ are entirely electrically isolated from each other and any conductive features of the lead 10, pulse generator 15 or patient. In other words, the shield layers 115′, 115″ are not electrically grounded and are not electrically connected to each other.

In other embodiments, the shield layers 115′, 115″ are each electrically coupled to a ground such as the pulse generator 15 or the patient. For example, as depicted in FIG. 7, in one embodiment, the proximal end 20 of the lead body 30 includes ground electrodes 140′, 140″. In one embodiment, the ground electrodes 140′, 140″ are in the form of rings extending about the outer circumferential surface of the lead body 30. The proximal ends of the shield layers 115′, 115″ are electrically coupled to the ground electrodes 140′, 140″, and the rest of the shield layers 115′, 115″ are electrically insolated from each other and the conductive features of the lead 10, pulse generator 15 and patient via electrical insulation. In one embodiment, the ground electrodes 140′, 140″ are located on the proximal end 20 of the lead body 30 just distal of the lead connective end 35 such that the ground electrodes 140′, 140″ are located just outside of the header 32 of the pulse generator 15 when the lead connective end 35 is received in the receptacle 33 of the header 32. When the lead 10 and pulse generator 15 are implanted in the patient, the ground electrodes 140′, 140″ are placed in electrical contact with the patient tissue surrounding the implanted pulse generator 15. In one embodiment, the shield layers 115′, 115″ do not have separate ground electrodes 140′, 140″, but instead share a common ground electrode 140.

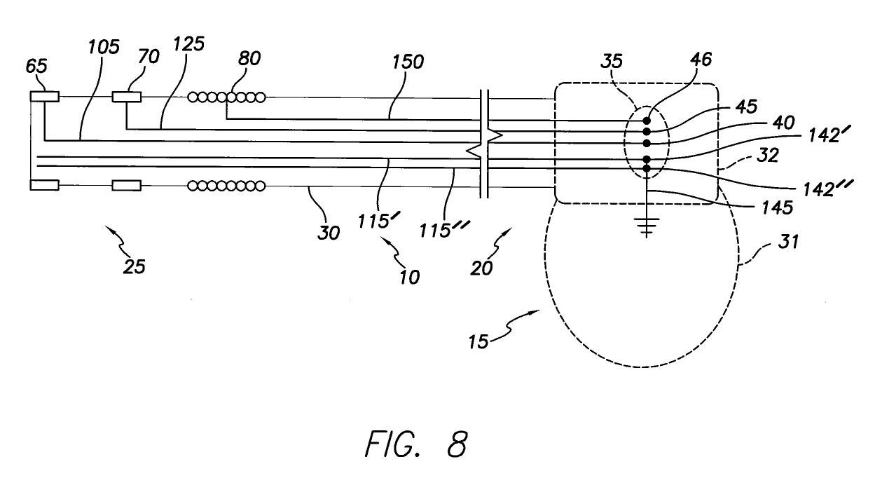

As depicted in FIG. 9, in one embodiment, the proximal ends of the shield layers 115′, 115″ are electrically coupled to respective contact rings 142′, 142″ on the lead connective end 35, and the rest of the shield layers 115′, 115″ are electrically insolated from each other and the conductive features of the lead 10, pulse generator 15 and patient via electrical insulation. When the lead connective end 35 is received in the receptacle 33 of the header 32 to electrically couple the lead 10 to the pulse generator 15, the contact rings 142′, 142″ are electrically coupled to a ground 145 in the pulse generator 15.

As illustrated in FIG. 6, in one embodiment, the outer insulation layer 130 extends about the outer circumferential surface of the second shield layer 115″. The outer insulation layer 130 extends generally the full length of the tubular body 30 between the proximal and distal ends 20, 25 of the body 30. The outer insulation layer 130 is generally tubular or cylindrical in shape. The outer insulation layer 130 is formed from an electrically insulative material such as Silicone, Optim, PTFE, Polyurethane, Carbosil, etc. The outer insulation layer 130 has a wall thickness of between approximately 0.004 inches and approximately 0.010 inches.

In one embodiment, the inner and outer conductor coils 105, 125 are helically wound in the same direction. However, in other embodiments, as can be understood from FIG. 6, the inner and outer conductor coils 105, 125 are helically wound in opposite directions. In either case, the first shield layer 115′ is helically wound in an opposite direction from the helically wound second shield layer 115″. The shield layers 115′, 115″ combine to act as a shield to the prevent the RF energy from a MRI from inducing current in the inner and outer conductor coils 105, 125. For example, in one embodiment, the shield layers 115′, 115″ act as an antenna for the field energy that would otherwise induce current in the conductor coils 105, 125. Additionally, because the shield layers 115′, 115″ are oppositely wound from each other, the RF field induced currents in the shield layers flow in opposing helical paths. As a result, the electric field induced within each shield layer due to their induced current oppose and cancel each other out. As a result, the RF field of the MRI remains invisible to the conductor layers 105, 125, and the possibility of currents being induced in the conductor layers 105, 125 is removed.

As can be understood from FIGS. 7 and 8, a conductor 150 extends through the tubular body 30 to electrically couple the defibrillation coil 80 to the contact ring 46. In one embodiment, the conductor 150 is a helical conductor coil similar to those described with respect to the inner and outer conductor coils 105, 125. In other embodiments, the conductor 150 is a wire or cable formed of electrically conductive materials similar to those employed for the inner and outer conductor coils 105, 125.

FIG. 6 depicts inner and intermediate insulation layers 110,120′, 120″ electrically isolating the inner conductor coil 105 from the outer conductor coil 125, the outer conductor coil 125 from the first shield layer 115′, and the shield layers 115′, 115″ from each other. However, in other embodiments, one or more of the insulation layers 110, 120′, 120″ can be eliminated where: one or more of the conductor coils 105, 125 and/or the shield layers 115′, 115″ are coated or jacketed with an electrical insulation; or the wires or filars forming the conductor coils 105, 125 and/or the shield layers 115′, 115″ are individually coated or jacketed with an electrical insulation. In other words, in one embodiment, one or more of the insulation layers 110, 120′, 120″ can be eliminated where other steps are taken to electrically insulate the conductor coils 105, 125 from each other and/or from the shield layers 115′, 115″.

While the inner and outer conductors 105, 125 are depicted in FIG. 6 as being coiled conductors, in other embodiments, one or both of the conductors 105, 125 can be multi-strand conductor cables or wires. For example, as depicted in FIG. 9, which is the same view depicted in FIG. 6, except of a fourth embodiment of the lead body, the inner conductor 105 is in the form of a helical coil, and the outer conductor 125 is in the form of a multi-strand cable. In other embodiments, the outer conductor 125 is in the form of a helical coil, and the inner conductor 105 is in the form of a multi-strand cable. In yet other embodiments, both conductors 105, 125 will be in the form of multi-strand cables. In various embodiments, regardless of the configuration of the conductors 105, 125, the rest of the lead body configuration is generally the same as discussed with respect to FIG. 6.

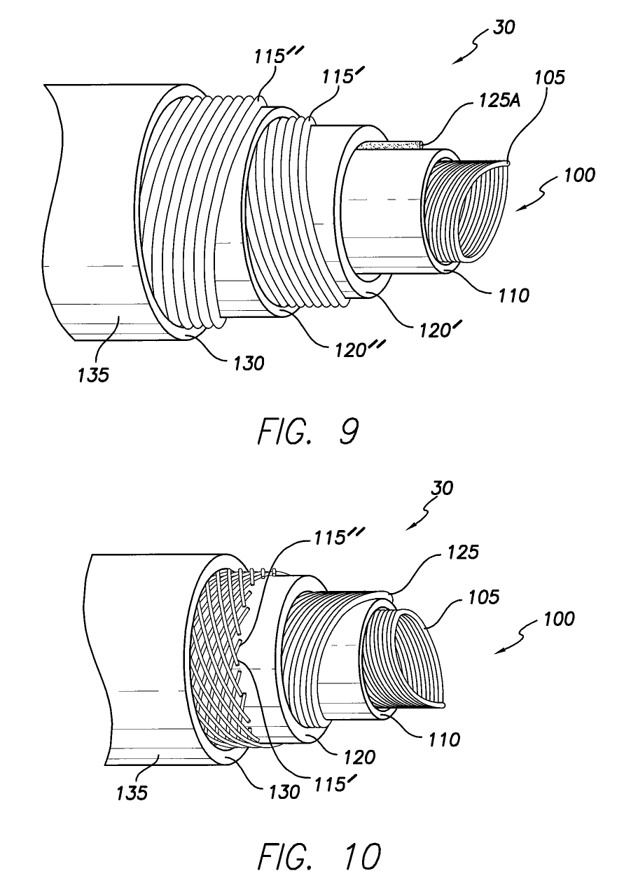

While the first and second shield layers 115′, 115″ are depicted in FIG. 6 as being separated from each other by the second intermediate insulation layer 120″, as mentioned above, the second intermediate insulation layer 120″ can be eliminated such that the first and second shield layers 115′, 115″ generally exist as one layer in the lead body 30. For example, as illustrated in FIGS. 10 and 11, which are both the same view depicted in FIG. 6, except of fifth and sixth embodiments of the lead body 30, the second intermediate insulation layer 120″ has been eliminated and the shield layers 115′, 115″ have been generally combined into a single shield layer 115 sandwiched between an intermediate insulation layer 120 and the outer insulation layer 130. In such embodiments, one or both shield layers 115′, 115″ may be formed of wires or filars that are insulated with an electrically insulative material such as PTFE, ETFE, PFA, Polyurethane, Optim etc.

As indicated in FIG. 10, in one embodiment, the windings of the first shield layer 115′ are helically wound over the outer circumferential surface of the intermediate insulation layer 120 in a first direction, and the windings of the second shield layer 115″ are helically wound over the first shield layer 115′ in a second direction. As shown in FIG. 11, in one embodiment, the windings of the shield layers 115′, 115″ are helically wound in opposite directions about the outer circumferential surface of the intermediate insulation layer 120 in a weaved or mesh fashion. Regardless of whether the shield layer 115 is configured with one shield layer 115″ wound over the other shield layer 115′ or the shield layers 115′, 115″ being weaved together, the shield layers 115′, 115″ perform in a similar fashion with respect to RF field induced currents as discussed with respect to the embodiment depicted in FIG. 6.

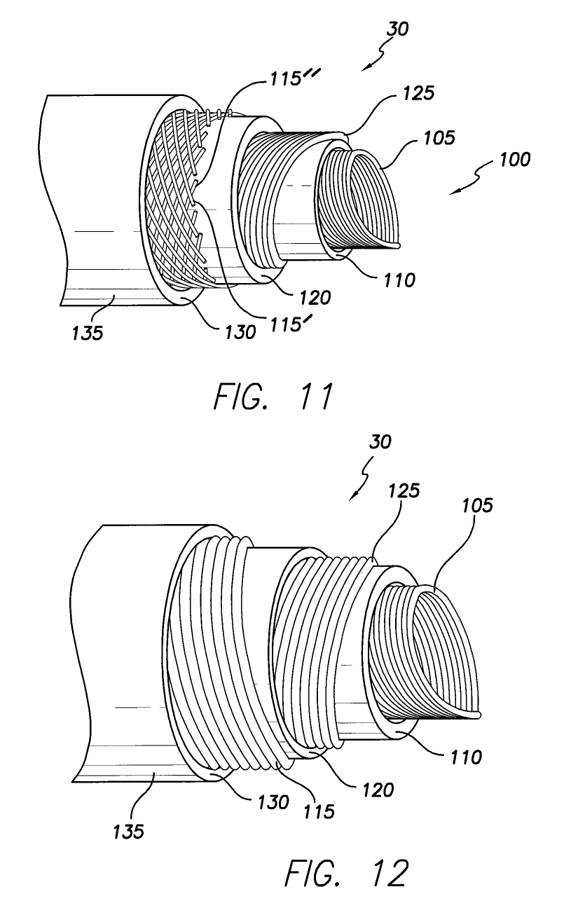

For a discussion of a seventh embodiment of the lead 10 configured to reduce, if not totally eliminate, the current induction and heating caused in lead conductors subjected to MRI, reference is made to FIG. 12. FIG. 12 is the same view depicted in FIG. 6, except of another embodiment of the lead body 30.

The preceding discussion regarding FIG. 6 and pertaining to the shield, conductor and insulation materials is generally applicable to the embodiment depicted in FIG. 12. Also, the preceding discussion regarding FIG. 6 and pertaining to the elimination of insulation layers via insulating the wires or filars of the conductors and/or shield is also generally applicable to the embodiment depicted in FIG. 12.

As shown in FIG. 12, in one embodiment, the shield layer 115 is a single helical coil wound in a single direction about the outer circumferential surface of the intermediate insulation layer 120 and covered by the outer insulation layer 130. The outer conductor 125 is sandwiched between the inner and intermediate insulation layers 110, 120, and the inner insulation layer 110 separates the outer conductor 125 from the inner conductor 105.

As can be understood from FIG. 12, in one embodiment, the outer of the two conductors 105, 125 is a helically wound coil conductor that is wound in a direction opposite from the wind direction of the helically wound shield layer 115. The shield layer 115 coupled with the outer conductor coil 125 acts as a shield to prevent the RF energy from a MRI from inducing current in the conductor coil 105. For example, RF field induced currents in the shield layer and outer conductor coil flow in opposing helical paths. As a result, the electric field induced these layers due to their induced current oppose and cancel each other out. As a result, the RF field of the MRI remains invisible to the conductor layer 105, and the possibility of currents being induced in the conductor layers 105 is removed. This configuration eliminates the added bulk of a second shield layer but still provides adequate shielding to the inner conductor layer and electrode which is most susceptible to the RF induced heating effect.

While the outer conductor 125 is a helically wound coil conductor wound oppositely from the helically wound shield layer 115, the inner conductor 105, if it exists, may be: a helically wound coil conductor 105, 125 (see FIG. 12) wound in the same direction or oppositely from the helically wound shield layer 115; or a wire or multi-strand conductor cable similar to that depicted as 125a in FIG. 9 may be substituted for the inner helically wound coil conductor 105 in FIG. 12. The outer conductor coil 125 will be wound oppositely from the helically wound shield layer 115.

In one embodiment, the shield layer 115 is entirely encapsulated via electrical insulation such that the shield layer 115 is entirely electrically isolated from any conductive features of the lead 10, pulse generator 15 or patient. In other words, the shield layer 115 is not electrically grounded. In other embodiments, the shield layer 115 is electrically coupled to a ground such as the pulse generator 15 or the patient, as depicted in, and discussed with respect to, FIGS. 3 and 4.

The embodiments discussed above with respect to FIGS. 1-12 depict lead bodies 30 with one or two conductors 105, 125 extending therethrough and electrically connected to electrodes 65, 70 near the distal end 25 of the lead 10. However, the concepts taught herein regarding shielding and induced current cancellation and reduction can be applied to other embodiments of the lead body 30 having less than or greater than two conductors 105, 125. Also, in various embodiments, the above-described shield configurations may be combined. For example, the mesh shield configuration depicted in FIGS. 2 and 5 may be combined in the same or separate layers with the wound shield configurations depicted in FIGS. 6 and 9-12.

For each of the embodiments discussed above with respect to FIGS. 1-12, the impedance of the coil conductors 105, 125 can be further impacted to reduce the current inductance in the conductors 105,125 via exposure to MRI. In one embodiment, reduction in current inductance may be achieved via impedance matching for the tissue-electrode interface and for the lead attached to the pulse generator.

It can be challenging to hit optimal values for both non-resonant length for MRI scans and appropriate tip size due to the dependence of effective length and lead tip area. To meet this challenge, in one embodiment, the impedance matching may be done by using coated conductors and by making them co-radial in order to hit the right impedance values for the pacing system. Due to the complexity of the terminal end, in one embodiment, a distributed lumped model may be used.

In one embodiment, the lead body 30 includes conductor coils 105, 125 that are co-radial, wherein the inner conductor coil 105 is coated to change the impedance of the system and enabling changes in the effective length of the lead conductors. For example, a co-radial coil embodiment may involve a multi-filar coil, wherein each filar may be individually insulated from its adjacent filars and may be used as an independent conductor. Where such a coil is a four filar coil with four individually insulated filars, the coil may have 4 conductors and may feed four different electrodes.

In one embodiment, there may be a co-radial coil that feeds both electrodes, for example, wherein a coil is a four filar coil and two filars drop off at a first electrode and the other two filars continue distally to a more distal electrode. Such a configuration changes the impedance of each conducting path. Also, the pitch of each conducting path becomes much bigger. By controlling the pitch, the impedance of the system, and the electrode-tissue surface interface, the system inductance may be tuned away from typical RF frequencies used in MRI (e.g., 1.5T, 3T, etc.), pulled away from resonance conditions within the MRI-lead system, and significantly dampen the induced fields/currents and their effects in the pacing system.

For the above-described shield-equipped embodiments, the shield layer(s) 115 is configured to reduce an amount of current induced in the electrical conductor(s) 105, 125 when present in an electromagnetic field as compared to the current that would be induced in the electrical conductor(s) 105, 125 absent the shield layer(s) 115. Similarly, for the embodiments employing oppositely wound coil conductors 105, 125, the coil conductors 105, 125 are configured to reduce an amount of current induced in the conductors 105, 125 when present in an electromagnetic field as compared to the current that would be induced in the conductors 105, 125 were the conductors 105, 125 not oppositely wound.

Although the present invention has been described with reference to preferred embodiments, persons skilled in the art will recognize that changes may be made in form and detail without departing from the spirit and scope of the invention.

What is claimed is:

1. An implantable medical lead for coupling to an implantable pulse generator and configured for improved MRI safety, the lead comprising: a tubular body including a proximal end and a distal end; a first electrode operably coupled to the tubular body near the distal end; a first electrical conductor extending distally through the body from the proximal end and electrically connected to the first electrode; and a pair of shield layers extending through the tubular body between the proximal and distal ends and configured to reduce an amount of current induced in the electrical conductor when present in an electromagnetic field as compared to the current that would be induced in the electrical conductor absent the shield layers.

2. The lead of claim 1, wherein at least one of the shield layers is a generally cylindrical mesh or weaved tube.

3. The lead of claim 1, wherein both shield layers are generally cylindrical mesh or weaved tubes.

4. The lead of claim 2, wherein the mesh or weaved tube is formed of helically wound wires or filars that are oppositely wound and in electrical contact with each other.

5. The lead of claim 1, wherein at least one of the shield layers is a helical coil.

6. The lead of claim 1, wherein the pair of shield layers is a pair of helical coils oppositely wound from each other.

7. The lead of claim 6, further comprising a tubular insulation layer separating the coils.

8. The lead of claim 6, wherein each coil includes wires or filars and the wires or filars of at least one of the coils have an electrical insulation jacketing or coating, and the coils generally form a single radial layer of the lead body.

9. The lead of claim 8, wherein one coil is wound directly over the other coil.

10. The lead of claim 8, wherein the coils are weaved through each other.

11. The lead as in any of claims 1-10, wherein the first electrical conductor is located radially inward of the most radially inward of the shield layers.

12. The lead as in any of claims 1-10, further comprising a second electrode operably coupled to the tubular body near the distal end and a second electrical conductor extending distally through the body from the proximal end and electrically connected to the second electrode.

13. The lead of claim 12, wherein both electrical conductors are located radially inward of the most radially inward of the shield layers.

14. The lead of claim 12, wherein the electrical conductors are helically wound conductors oppositely wound from each other.

15. The lead as in any of claims 1-10, wherein at least one of the shield layers is electrically grounded.

16. The lead of claim 15, wherein the at least one of the shield layers is electrically grounded by being electrically coupled to patient fluid or tissue.

17. The lead of claim 15, further comprising a grounding electrode operably coupled to the tubular body near the proximal end and electrically connected to the at least one of the shield layers.

18. The lead of claim 15, wherein the at least one of the shield layers is electrically grounded by being electrically coupled to the pulse generator.

19. An implantable medical lead for coupling to an implantable pulse generator and configured for improved MRI safety, the lead comprising: a tubular body including a proximal end and a distal end; a first electrode operably coupled to the tubular body near the distal end; a first electrical conductor extending distally through the body from the proximal end and electrically connected to the first electrode; and a shield layer located radially outward relative to the first electrical conductor and extending through the tubular body between the proximal and distal ends and configured to reduce an amount of current induced in the electrical conductor when present in an electromagnetic field as compared to the current that would be induced in the electrical conductor absent the shield layers.

20. The lead of claim 19, wherein the shield layer is a generally cylindrical mesh or weaved tube.

21. The lead of claim 19, wherein the shield layer is a pair of helically wound coils.

22. The lead of claim 19, wherein the first electrical conductor is a helically wound coil oppositely wound relative to the shield layer.

23. The lead of claim 19, further comprising a second electrical conductor and wherein the first and second electrical conductors are helical coils oppositely wound relative to each other.

24. The lead as in any of claims 19-23, wherein the shield layer is electrically grounded.

25. The lead of claim 24, wherein the shield layer is electrically grounded by being electrically coupled to patient fluid or tissue.

26. The lead of claim 24, further comprising a grounding electrode operably coupled to the tubular body near the proximal end and electrically connected to the shield layer.

27. The lead of claim 24, wherein the shield layer is electrically grounded by being electrically coupled to the pulse generator.
